(19) United States
(12) Patent Application Publication
(10) Pub. No.: US 2021/0233606 A1
(43) Pub. Date: Jul. 29, 2021

(54) MEASUREMENT AND PREDICTION OF VIRUS GENETIC MUTATION PATTERNS

(71) Applicant: The Chinese University of Hong Kong, Shatin, New Territories, Hong Kong (CN)

(72) Inventors: Maggie Haitian WANG, Shatin, New Territories, Hong Kong SAR (CN); Benny Chung Ying ZEE, Kowloon, Hong Kong SAR (CN); Jingzhi LOU, Hangzhou, Zhejiang (CN); Ka Chun CHONG, Ma On Shan, N.T., Hong Kong SAR (CN)

(21) Appl. No.: 17/252,698

(22) PCT Filed: Jun. 18, 2019
(86) PCT No.: PCT/CN2019/091652

§ 371 Date: Dec. 15, 2020

Related U.S. Application Data
(60) Provisional application No. 62/687,645, filed on Jun. 20, 2018.

(51) Int. Cl. G16B 5/30; G16B 20/20; G16B 20/50; G06F 30/20; G16H 50/50; G16H 10/40; G16H 50/80

(52) U.S. Cl. CPC G16B 5/30; G16B 20/20; G16B 20/50; G16H 50/80; G16H 50/50; G16H 10/40; G06F 30/20

(57) ABSTRACT

Mutation patterns of a virus (e.g., influenza virus) are identified and predicted based on identifying effective mutations in an amino acid sequence of the virus and an effective mutation period during which the mutation enables the virus to escape from human immunity. Based on analysis of existing virus composition and infection rates, a measure of genetic mutation activity (“g-measure”) is determined, and one or more associated parameters that further characterize virus genetic activity may also be optimized. The g-measure and/or associated parameters can be used to predict future genetic activity of the virus, which can aid in selection of strains for a future vaccine and/or predictions of infectious-disease outbreaks.

### CROSS-REFERENCE TO RELATED APPLICATIONS

This application claims the benefit of U.S. Provisional Application No. 62/687,645, filed Jun. 20, 2018, the disclosure of which is incorporated by reference in its entirety.

### BACKGROUND

The present disclosure relates generally to genetic epidemiology of viral infectious diseases (e.g., influenza) and in particular to measurement and prediction of virus genetic (or amino acid) mutation patterns for viruses that cause infectious diseases.

Influenza, also referred to as “flu,” is a contagious respiratory ailment that has plagued humanity for centuries. When it was discovered that flu is caused by a virus (the influenza virus, or flu virus), hope for an effective vaccine rose, and after years of research, flu vaccines are now widely available. However, the flu virus mutates rapidly into new strains, and a vaccine that is effective against one strain may not be effective against other (mutated) strains. Accordingly, the “recipe” of flu virus strains used in preparation of flu vaccines is regularly modified based on predictions about future effective strains, and individuals are encouraged to obtain a new flu vaccine annually, in an effort to help their immune systems keep up with the mutating flu virus.

The present protocol for production and distribution of flu vaccines involves deciding each year which flu-virus strains to protect against in the next iteration of the vaccination. At present, this decision is based on samples of flu virus from around the world, known antigenic sites (e.g., specific amino acids in the viral sequence), and lessons about viral mutation patterns learned from experience, with the goal being to predict which strains of flu virus will be effective against human immune systems (i.e., disease-producing) at the time when the new vaccine is ready, typically about eighteen months to two years in the future. The flu vaccine is prepared according to this prediction.

The predictions are not always accurate, and as a result, flu vaccines vary widely in effectiveness from year to year. This in turn makes individuals less likely to make the effort to obtain a flu vaccination, which compromises the “herd immunity” effect that is achieved when most people are immunized against an infectious agent.

Improved techniques for predicting virus mutations, and in particular for predicting which mutations will be effective against human immune systems in a future time frame of at least two years, would therefore be useful.

### SUMMARY

Certain embodiments of the present invention relate to techniques for measurement and prediction of virus mutation patterns based on viral sequences (e.g., amino acid sequences) and population epidemic level. The predictions are based on identifying an “effective mutation,” i.e., a mutation (variation in an amino acid sequence or nucleic acid sequence) that contributes to the virus's evolutionary advantage over human immunity, as opposed to a “trivial mutation” that has no (or negligible) effect on the virus's ability to survive and reproduce. The predictions are also based on an assumption that human immunity will eventually learn to recognize and block an effective mutation (either with or without the aid of a vaccine). This implies that an effective mutation has an “effective mutation period,” which is the time interval during which the mutation enables the virus to escape from human immunity. Identifying effective mutations and determining the effective mutation period, using techniques described herein, allows for improved predictions of which strains of a given virus (i.e., which mutations) will be prevalent in future time periods. Such predictions can be used for a variety of practical purposes, including: (1) aiding in selection of viral strains for vaccine production; (2) providing real-time information about the likely efficacy of a given version of a vaccine; and/or (3) forecasting virus activity (e.g., rates of occurrence of an infectious disease caused by the virus).

Some illustrative techniques used herein rely on analysis of a longitudinal cohort of flu virus composition (amino acid sequences) and infection rates to compute a measure of genetic mutation activity, referred to herein as “g-measure,” for the flu virus. The g-measure, described more specifically below, models at least two aspects of genetic activity. The first is whether a single mutation should be considered important. On the assumption that a more adaptive mutation will spread widely after newly appearing while an insignificant mutation will not, the prevalence of a single residue contributes to higher g-measure. The second aspect of genetic activity is the number of simultaneous mutations, which captures potential antigenetic shift with multiple residue substitutions at the same time; a higher number of effective mutations at a given prevalence will increase the g-measure. Accordingly, the g-measure reflects both the adaptiveness of mutations and the number of simultaneous effective mutations. Further, if a residue has more than one effective mutation period within the investigation period, the g-measure will encompass later effective mutation periods. Computing the g-measure also includes optimizing parameters that further characterize flu virus genetic activity, such as a dominance threshold (a minimum prevalence required for a residue to be considered as an effective mutation) and an extended effectiveness period (representing the time during which an effective mutation remains effective against human immunity after achieving dominance). The g-measure and/or associated parameters can be used to predict future genetic activity of the flu virus, which can aid in selection of strains for the next flu vaccine and/or predictions of flu outbreaks. Similar techniques can be applied to other viruses and associated infectious diseases.

The following detailed description, together with the accompanying drawings, provides a better understanding of the nature and advantages of the claimed invention.

### BRIEF DESCRIPTION OF THE DRAWINGS

FIGS. 1A-1C illustrate a simplified example of construction of coding sequences according to an embodiment of the present invention. FIG. 1A shows four example amino acid sequences observed during a time period. FIG. 1B shows a tag sequence that can be defined for the investigation period according to an embodiment of the present invention.

FIG. 1C shows coding sequences corresponding to the amino acid sequences of FIG. 1A and the tag sequence of FIG. 1B.

FIG. 1D shows a prevalence vector computed from the coding sequences of FIG. 1C according to an embodiment of the present invention.

FIG. 2 shows a simplified example of identifying effective mutations and effective mutation periods from prevalence vectors according to an embodiment of the present invention.

FIGS. 3 and 4 are graphs showing the correlation of g-measure with observed variations in flu infections in a population. FIG. 3 shows data obtained from observations of flu virus activity in Hong Kong between 1996 and 2015. FIG. 4 shows data obtained from observations of flu virus activity in New York between 2003 and 2016.

FIG. 5 shows a flow diagram of a process for measuring and predicting flu virus activity according to an embodiment of the present invention.

### DETAILED DESCRIPTION

Techniques for modeling virus activity described herein rely on analysis of a longitudinal cohort of virus composition (amino acid sequences) and infection rates to compute a measure of genetic mutation activity, referred to herein as “g-measure,” for the virus. The analysis is performed over an “investigation period” that is divided into a set of time periods of equal duration. In some embodiments, each time period can be a year; other embodiments may define shorter time periods (e.g., three months, one month, one week) or longer time periods (e.g., two years, five years, etc.). For purposes of illustration, reference is made to the influenza, or “flu,” virus; however, the techniques described can be applied to other viruses.

For a given time period t, a number nₜ of samples of the flu virus (or other virus of interest) are collected. For each sample i in time period t, an amino acid sequence {xᵢⱼ^{t}} for the virus is determined, where index j indicates a specific position within the amino acid sequence and x is an identifier of a specific amino acid. Amino acid sequences for a given sample of flu virus can be determined using conventional or other techniques, and a particular sequencing technique is not critical to understanding the present disclosure. In general, nₜ instances of {xᵢⱼ^{t}} are determined.

It is assumed that the virus may mutate during the investigation period and that different samples of flu virus collected within the same time period may have different mutations. To facilitate analysis of mutations, it is helpful to define a “tag sequence” for the investigation period that can be used to represent every sample in a uniform format. The tag sequence can be an amino acid sequence {aₖ} for k=1, . . . , K, where K is defined as:

$$K = {\sum }_{j = 1}^{J} {q}_{j} , \qquad ( 1 )$$

where J is the total amino acid sequence length for the virus, and qⱼ is the number of unique amino acids observed in position j across the investigation period. The tag sequence {aₖ} can be formed by concatenating all unique amino acids observed at each position j of the amino acid sequence. The tag sequence enables assessment of mutations without establishing a reference sequence (which is conventional practice); thus, rather than comparison of sequences, the tag sequence provides a tool to capture the dynamics of every possible residue.

Given the tag sequence {aₖ}, each observed amino acid sequence {xᵢⱼ^{t}} can be represented as a coding sequence Aᵢ^{t}. The coding sequence can be a sequence of K indicators (e.g., bits), one for each position kin the tag sequence; the indicator in the kth position can be set to a first value (e.g., 1) if the corresponding amino acid at position j is present in sample i and to a second value (e.g., 0) if not.

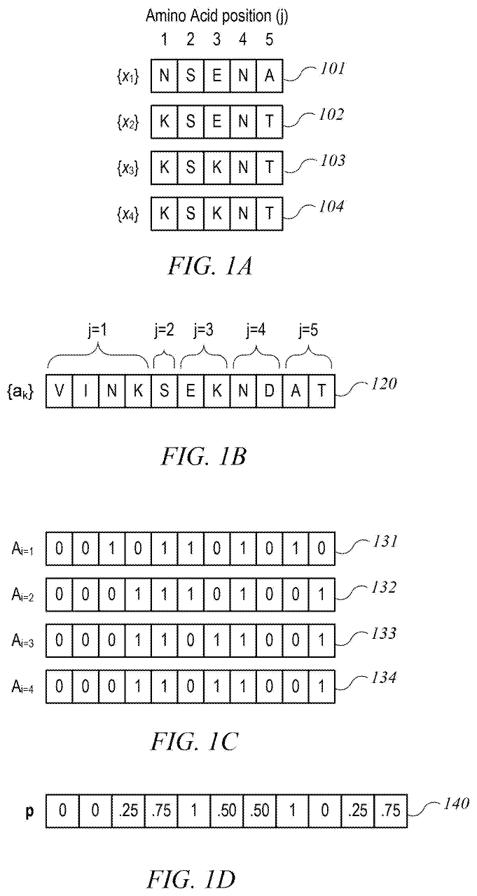

FIGS. 1A-1C illustrate a simplified example of construction of coding sequences Aᵢ^{t} according to an embodiment of the present invention. FIG. 1A shows four example amino acid sequences 101, 102, 103, 104 observed during a time period t (e.g., one year); amino acids are denoted by one-letter codes using the standard IUPAC one-letter coding scheme. As can be seen, in the observed sequences 101-104 the first (j=1) position has amino acid N or K; the second (j=2) position has amino acid S; the third (j=3) position has amino acid E or K; the fourth (j=4) position has amino acid N; and the fifth (j=5) position has amino acid A or T.

In this example, it is assumed that amino acid sequences are also observed in other time periods (e.g., years) during the investigation period and that other amino acids are observed for some of the positions in at least some of those time periods. Specifically, it is assumed that the following observations are made: for position j=1, amino acid V, I, N, or K; for position j=2, amino acid S; for position j=3, amino acid E or K; for position j=4, amino acid N or D; and for position j=5, amino acid A or T. FIG. 1B shows a tag sequence 120 that can be defined for the investigation period according to an embodiment of the present invention. In this example, the bits of tag sequence 120 are ordered such that the first four tag-sequence positions correspond to amino acids observed at the j=1 position, the next tag-sequence position to the j=2 position, and so on. Where multiple bits of the tag sequence correspond to the same position in the amino acid sequence, the bits can be ordered based on time period of first observation. Other orderings can be used if desired.

FIG. 1C shows coding sequences 131, 132, 133, 134 corresponding to amino acid sequences 101, 102, 103, 104 respectively. Coding sequences 131-134 provide the same information as the original amino acid sequences 101-104 but in a format that facilitates computational analysis as described below. It should be understood that the amino acid sequence of a flu virus is much longer than in this simplified example and that the number of sequence samples obtained within a time period may be much larger than the four instances shown. It should also be understood that the specific sequences in FIGS. 1A-1C are merely for purposes of illustration and may or may not correspond to an existing virus.

Given a set of nₜ coding sequences Aᵢ^{t} corresponding to samples i observed during time period t, a prevalence vector p^{t}=(p₁^{t}, . . . , pₖ^{t}, . . . , p_{K}^{t}) for time period t can be defined as:

$${p}_{k}^{t} = {\sum }_{i = 1}^{{n}_{t}} {A}_{i k}^{t} / {n}_{t} \qquad ( 2 )$$

Each component of prevalence vector p^{t} can be understood as representing the prevalence of a particular amino acid at a particular position in the amino acid sequence. FIG. 1D shows a prevalence vector p^{t} computed from the coding sequences of FIG. 1C according to Eq. (2).

Prevalence vectors p^{t} can be analyzed across the time periods within the investigation period in order to identify effective mutations, i.e., mutations that provide an evolutionary advantage against human immunity. A mutation can be identified by detecting a change in prevalence at tag position k from zero at time period t⁰ to nonzero at subsequent time period(s) t⁰+1, etc. It is assumed that effective mutations will increase in prevalence and eventually reach at least a threshold prevalence, referred to herein as the “dominance threshold” and denoted as θ. For purposes of analysis, a mutation at position aₖ of the tag sequence is defined as effective if there exists, within the investigation period, a time t⁰ and a time t^{θ} such that:

pₖ^{t}^{⁰}=0,pₖ^{t}^{⁰}⁺¹>0, and pₖ^{t}^{^{θ}}≥θ. (3)

As described below, the value of dominance threshold θ can determined empirically.

It is also useful to define an effective mutation period (EMP, denoted herein by ω), which represents the length of time that an effective mutation retains its evolutionary advantage. This period includes the transition time t^{θ}−t⁰ (i.e., the time from first appearance of the mutation to the time the mutation reaches the dominance threshold). The EMP also includes an “extended effective mutation period,” denoted h, which corresponds to the length of time that the mutation retains its evolutionary advantage after reaching dominance. Thus, for a given mutation at position k, the total EMP is defined as:

ωₖ(θ,h)={t⁰<t≤t^{θ}+h|θ,h,k}. (4)

The set of effective mutations at time period t (denoted herein by W^{t}) is:

W^{t}={a^{t}ₖ,t∈ωₖ,k=1, . . . ,K}. (5)

Optimal values of θ and h can be determined empirically using a fitting procedure described below. In principle, the values of θ and h may be specific to a particular position k in the tag sequence {aₖ}; however, in practice it may not be feasible to gather enough data to determine a per-position fit, and it may be assumed that all mutations share the same values of θ and h. In one specific example, θ=0.8 and h=2.

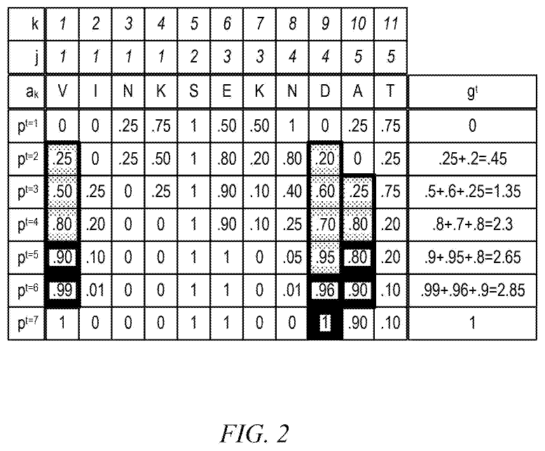

FIG. 2 shows a simplified example of identifying effective mutations and EMP from prevalence vectors according to an embodiment of the present invention. The tag sequence {aₖ} from FIG. 1B is assumed, and the prevalence vector p of FIG. 1D is assumed to be the prevalence vector for time period t=1. Prevalence vectors p^{t} for additional time periods t=2 through t=7 are shown; these vectors can be determined in the manner described above. For purposes of illustration, it is assumed that θ=0.8 and h=2. For each effective mutation (i.e., a mutation satisfying the conditions of Eq. (2)), the prevalence values are highlighted in light gray for the transition time and in black for the extended effective mutation period. The total EMP is outlined in heavy black lines. It should be noted that although the values of θ and h are assumed to be position-independent, the total EMP can vary due to differences in transition time. The mutations at positions k=6 and k=8 are not identified as effective mutations in this analysis, even though they do satisfy the dominance threshold in at least some time periods, because the transition from zero prevalence to nonzero prevalence occurs prior to t=1.

After identifying the effective mutations and EMP for each, a measure of genetic mutation activity (referred to herein as “g-measure”) can be defined. Specifically, for each time period t a K-component indicator vector m^{t} is defined as:

$${m}_{k}^{t} ( θ , h ) = \{ \begin{matrix}1 & t ∈ ω ( θ , h ) \\ 0 & otherwise\end{matrix} , \qquad ( 6 )$$

where ω(θ, h) is defined according to Eq. (4). The g-measure can be defined as:

$${g}^{t} ( θ , h ) = {m}^{t} \cdot {p}^{t} = {\sum }_{k = 1}^{K} {m}_{k}^{t} ( θ , h ) {p}_{k}^{t} . \qquad ( 7 )$$

In FIG. 2, g^{t} computed according to Eq. (7) is shown for each time period. A g-measure vector g=[g^{t}] represents the trend of mutation activity across time periods.

The g-measure can be understood as a function (e.g., sum) of prevalence of all effective mutations for a given time period. This models two relevant aspects of genetic activity. The first is whether a mutation should be considered important. On the assumption that a more adaptive mutation will spread widely after newly appearing while an insignificant mutation will not, the prevalence of a single residue contributes to higher g-measure. The second aspect of genetic activity is the number of simultaneous mutations, which captures potential antigenetic shift with multiple residue substitutions at the same time; a higher number of effective mutations at a given prevalence will increase the g-measure. Accordingly, the g-measure reflects both the adaptiveness of mutations and the number of simultaneous effective mutations. Further, if a residue has more than one effective mutation period within the investigation period, the g-measure will encompass all effective mutation periods. The g-measure can be used for various purposes, including: (1) predicting epidemiology; (2) selecting component amino acids for the next flu vaccine based on effective mutations and EMPs; (3) evaluating a currently available flu vaccine strain based on comparing currently effective mutations to the vaccine strain.

As described above, the g-measure is dependent on two parameters: the dominance threshold θ and the extended effective mutation period h. In some embodiments, values for these parameters can be determined empirically based on a population-level epidemic variable, such as seropositivity rate of a subtype, the number of diagnosed cases of viral infection within a time period or the rate of hospitalization for viral infection within the time period. It is expected that time variation in the g-measure should correlate with time variations in the population-level epidemic variables, because the spread of a new effective mutation would result in more infections in the population.

Accordingly, in some embodiments of the present invention, the following fitting procedure can be used to determine values of θ and h. A population-level epidemic variable (e.g., number of diagnosed cases or number of hospitalizations) is defined as a vector f=[ƒ^{t}], where index t denotes one of the time periods in the investigation period. A function S(f, g) that measures the quality of matching between vectors g and f is chosen. For example, S can be the p-value of a goodness-of-fit statistic for a generalized linear model in which f is the response variable and g is the predictor variable. In this case, a smaller value of S indicates a better match between the response and the predictor. Optimal values of θ and h can be defined as the values (\hat{θ}, ĥ) that minimize S, i.e.:

$$( \hat{θ} , \hat{h} ) = {argmin}_{θ ∈ Θ , h ϵ H} \{ S ( f , g \vert θ , h ) \} , \qquad ( 8 )$$

where H={0, 1, 2, . . . } and Θ=[0.5, 1].

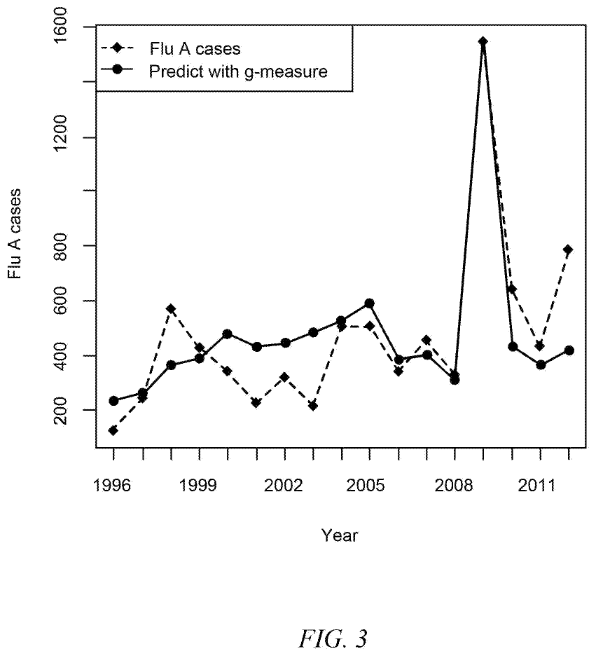

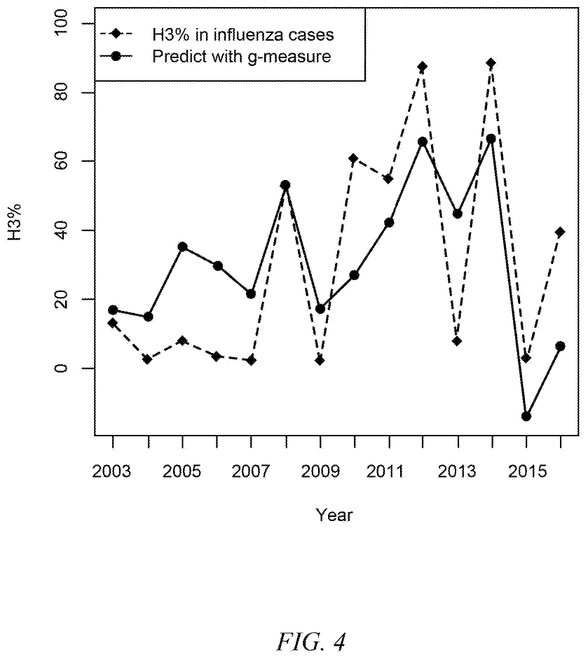

By way of illustration, FIGS. 3 and 4 are graphs showing the correlation of g-measure with observed variations in flu infections in a population. FIG. 3 shows data obtained from observations of flu virus activity in Hong Kong between 1996 and 2015. The diamond data points connected by dashed lines correspond to the number of cases of influenza A diagnosed each year. The round data points connected by solid lines represent the number of cases predicted using the g-measure computed as described above. Similarly, FIG. 4 shows data obtained from observations of flu virus activity in New York between 2003 and 2016. The diamond data points connected by dashed lines show the percentage of influenza cases in a given year that were attributed to H3 strains of the virus. The round data points connected by solid lines represent the number of such cases predicted using the g-measure computed as described above. As can be seen from FIGS. 3 and 4, the g-measure, with optimal values of θ and h can model variations in incidence of flu in a population.

A g-measure as described herein can be used to make predictions regarding future flu virus activity. In some embodiments, predictions of future incidence of flu can be made. For example, if the fitting function S(f, g) is the p-value of a goodness-of-fit statistic of a Poisson regression model, then the following fitted model can be obtained from existing data:

log(ƒ|X,\hat{θ},ĥ)=\hat{β}₀+\hat{β}₁g(\hat{θ},ĥ)+\hat{β}₂X+\hat{β}₃T, (9)

where X are environmental covariates related to epidemics (e.g., temperature and humidity) and T is a time variable; coefficients \hat{β}₀ to \hat{β}₃ are determined by fitting. More complicated fitting functions, such as system dynamic models, can also be used when sample size is sufficient.

When virus sequence samples for time period t+1 are available, the g-measure can be computed according to Eq. (7), using p^{t+1} and (\hat{θ}, ĥ). When sequence samples are not available (e.g., when t+1 corresponds to a future time period), p^{t+1} can be prospectively estimated based on the conditional prevalence distribution Pr(pₖ^{l}|pₖ^{l-1}) (l=1, . . . , t) in existing data; the estimate of prevalence at time period t+1 is:

\hat{p}ₖ^{t+1}=E(pₖ^{l}|pₖ^{l-1},k=1, . . . ,K,l=1, . . . ,t), (10)

where E denotes an expectation value determined from the conditional prevalence distribution Pr(pₖ^{l}|pₖ^{l-1}). Predictions for m^{t+1} and g^{t+1} can be computed from p^{t+1} in the manner described above, and the predicted epidemic level is given by:

\hat{ƒ}^{t+1}=exp[\hat{β}₀+\hat{β}₁ĝ^{t+1}+\hat{β}₂X^{t+1}+\hat{β}₃(t+1)]. (11)

In some embodiments, prediction of the next dominant influenza subtype can be made. For example, g-measures can be obtained for each subtype, and the one with the highest ĝ^{t+1} is the predicted dominant subtype for the next time period. In general, variations of g-measure, i.e., functions based on mutation prevalence, can be used to predict the next dominant subtype and other future flu trends.

In some embodiments, predictions of effective mutations can also be made. Eq. (5) defines the set of effective mutations W^{t} for time period t. Predictions for Wₜ₊₁ can be made starting from W^{t}. Eq. (10) and the dominance threshold \hat{θ} can be used to identify mutations likely to become dominant in time period t+1. Extended EMP ĥ can be used to identify effective mutations in W^{t} that are likely to lose effectiveness in time period t+1. The predicted set of effective mutations W^{t+1} can be used in vaccine antigen design. For instance, for vaccines that use genetically engineered residues, W^{t+1} identifies the amino acids to include.

In some embodiments, a representative viral sequence {zⱼ^{t}} can be defined for time period t. For example, for each amino acid position j, the amino acid with highest prevalence at that position can be identified as representative. By way of illustration, referring to the tag sequence of FIG. 1B and the prevalence vector of FIG. 1D, for position j=1, amino acid K has the highest prevalence (p=0.75); for position j=2, amino acid S has the highest prevalence (p=1); for position j=3, amino acids E and K have the same prevalence (p=0.5) so either can be chosen; for position j=4, amino acid N has the highest prevalence (p=1); and for position j=5, amino acid T has the highest prevalence (p=0.75). More generally, as described above, tag sequence {aₖ} includes a number qⱼ of amino acids corresponding to each position in the amino acid sequence. In that case, each element of representative viral sequence {zⱼ^{t}} would be:

zⱼ^{t}=aⱼ₊ᵣ_{₀}₋₁, (12)

where r₀ is the value of an index r that yields:

$${max}_{{r}_{L} < r \leq {r}_{U}} {p}_{{r}_{L} + r}^{t} , \qquad ( 13 )$$

where, for sequence position j, the range (r_{L}, r_{U}] is defined by:

$$\begin{matrix}{r}_{L} = {\sum }_{u = 1}^{j - 1} {q}_{u} , & ( 14 a ) \\ {r}_{U} = {r}_{L} + {q}_{j} . & ( 14 b )\end{matrix}$$

The representative viral sequence {zⱼ^{t}} is a probabilistic summary of the virus that naturally includes all effective mutations at time t. Comparing the representative viral sequence to strains included in a currently available flu vaccine allows assessment of the likely effectiveness of the vaccine. For instance, a distance can be computed between the representative viral sequence {zⱼ^{t}} and strains included in currently available flu vaccines. For this purpose, distance can be defined according to a conventional similarity measure for sequences, such as the p-distance or Hamming distance for amino acids. The smaller the distance, the better the match (and the more effective the vaccine is likely to be for protecting patients from flu infection).

In some embodiments, a representative viral sequence {zⱼ^{t+1}} for a future time period can be defined in the same manner using the prospective prevalence vector defined at Eq. (10) above. Where flu vaccine is prepared from existing wild-type virus, an optimal candidate virus for the next vaccine may be selected by identifying the existing wild-type virus that has closest distance to the predicted representative viral sequence {zⱼ^{t+1}}. As noted above, distance can be defined according to a conventional similarity measure for sequences, such as the p-distance for amino acids. When a predicted effective mutation of the representative viral sequence is not found in the wild-type strain, genetic engineering techniques can be applied to the wild-type sequence to make it exactly the same or as similar as possible to the predicted sequence.

The analytical approach described herein can be applied to sequence and epidemic data for a specific region, to global data, or to a mathematical combination of regional and global data. The prediction for a candidate vaccine virus can be specific to a particular region (e.g., country, continent, or hemisphere) or made for global use.

The analytical approach described herein can be applied to any or all gene segments of an influenza virus. Since each gene may have different θ and h parameters, the fitting of multiple g-measures for many genes can be carried out simultaneously when the sample size is large enough (global estimation), or the θ and h parameters can be estimated for the important genes first (e.g., Hemagglutinin and Neuraminidase, the most commonly mutated segments) followed by conditionally estimating the θ and h parameters for the remaining gene segments (local optimization).

The analytical approach described herein can be applied to any influenza subtypes, such as H3N2, pandemic H1N1, B/Yamagata, B/Victoria. The same approach can also be applied to other known infectious-disease-causing viruses, such as the A-EV71 virus (cause of Hand-Foot-and-Mouth disease), rhinoviruses (cause of the common cold), or new emerging pathogens that may cause epidemics or pandemics.

The sequencing data employed in analysis of the kind described herein can be obtained using any available sequencing technologies, including but not limited to first-generation sequencing (Sanger), next-generation sequencing (Illumina platform), or third-generation sequencing (PacBio platform or Nanopore platform).

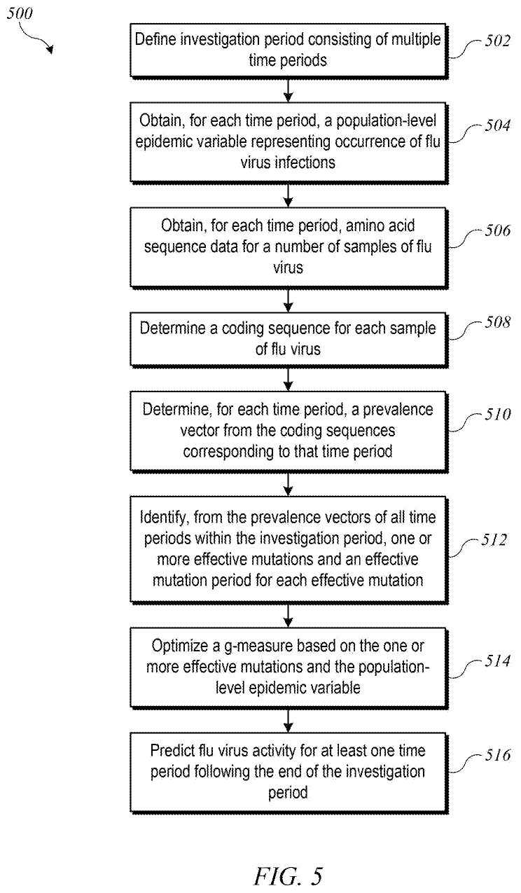

The analytical approach described herein can be employed in a computer-implemented method for predicting flu virus activity. FIG. 5 shows a flow diagram of a process 500 for measuring and predicting flu virus activity according to an embodiment of the present invention. FIG. 5 can be implemented, e.g., using a computer system of conventional design. Inputs to the process can include real-world data collected during an investigation period, including data about incidence or rates of reported cases of flu and sequence data for flu viruses observed during the investigation period.

At block 502, an investigation period is defined. The investigation period can be as long as desired, e.g., 10 years, 15 years, 20 years, or the like. The investigation period can be divided into a number of equal-length time periods (e.g., one-year periods, three-month periods, or the like). The selection of investigation periods and the length of each time period may be based on availability of data usable to determine prevalence of specific mutations in the flu virus.

At block 504, for each time period, a population-level epidemic variable is obtained. As described above, this can be a variable representing the number or frequency of occurrence of flu virus infections in people. Depending on what data sources are available, the population-level epidemic variable can be based on reported diagnoses of flu and/or reported hospitalizations for flu. Such data may be available in public health records going back many years. In addition or instead, sampling from a prospective longitudinal cohort may be used, and process 500 can be performed on any combination of data acquired retrospectively and/or from ongoing sampling.

At block 506, for each time period, amino acid sequences for samples of the flu virus are obtained. For instance, samples of flu virus may be periodically collected and sequenced. Samples may be collected from infected patients, from environmental surfaces, or in any other manner. An amino acid sequence for a sample of flu virus can be determined using conventional techniques. It is noted that obtaining and sequencing of flu virus has become routine practice in at least some parts of the world, allowing process 500 to be performed using previously- and presently-acquired and recorded data.

At block 508, a coding sequence for each sample of flu virus across all time periods is determined. As described above, the coding sequence can be determined by first generating a tag sequence representing every amino acid observed at each sequence position across the investigation period, and the coding sequence for a particular sample can be determined based on which of the observed amino acids are present in each sequence position for that particular sample.

At block 510, for each time period, a prevalence vector is determined from the coding sequences pertaining to that time period. The prevalence vector can be computed in the manner described above.

At block 512, based on the prevalence vectors for all of the time periods in the investigation period, one or more effective mutations can be identified, and, for each effective mutation, an effective mutation period can be identified. As described above, identification of an effective mutation can be based on whether the mutation first appears after the first time period and whether the mutation achieves a dominance threshold θ. The effective mutation period can be identified as the time from first appearance to reaching the dominance threshold plus an extended effective mutation period h.

At block 514, a g-measure is optimized based on the one or more effective mutations identified at block 512 and the population-level epidemic variable obtained at block 504. For instance, as described above, a similarity function S(f, g) can be defined such that smaller S indicates closer matching between f (the vector representing the observed population-level epidemic variable) and g. The vector g-measure can be computed using different combinations of values of θ and h, and for each g(θ, h) a value of S can be determined. By iterating over different combinations of values of θ and h, the values that minimize S can be determined.

At block 516, predictions of future flu virus activity (i.e., activity during at least one “future” time period t+1 following the last time period of the investigation period) are made. The predictions can be computed based on the g-measure and/or patterns observed in the prevalence vectors. Predictive methods described above can be used. For instance, future epidemic levels can be predicted using Eqs. (10) and (11). Future effective mutations can be predicted using Eq. (10) and the definition of effective mutations at Eq. (5). A future representative viral sequence can be predicted using Eqs. (10) and (12)-(14b). Vaccine match scoring can be based on distance between a current representative viral sequence (as described above) and viral strains included in the vaccine.

Predictions made at block 516 can be reported to medical professionals for various uses. Examples include: preparing for a predicted increase in flu infections (including issuing public health advisories, producing additional medications used to treat flu patients, etc.); selecting flu strains (wild-type or genetically engineered sequences) to include in a flu vaccine; and/or assessing likely effectiveness of currently available flu vaccines.

While the invention has been described with reference to specific embodiments, those skilled in the art will appreciate that variations and modifications are possible. All processes described above are illustrative and may be modified. Processing operations described as separate blocks may be combined, order of operations can be modified to the extent logic permits, processing operations described above can be altered or omitted, and additional processing operations not specifically described may be added. Particular definitions and data formats can be modified as desired.

The investigation period can be as long or short as desired, depending on availability of data. In some embodiments, the virus samples and population-level data can be localized to a particular area (e.g., a country, a state or region, a city), allowing for modeling of geographic variations in virus activity.

Further, while the embodiments described above refer specifically to the flu virus, those skilled in the art will appreciate that the same analytical approach can be applied to other viruses associated with other infectious diseases, and the invention is not limited to any particular virus.

Data analysis and computational operations of the kind described herein can be implemented in computer systems that may be of generally conventional design, such as a desktop computer, laptop computer, tablet computer, mobile device (e.g., smart phone), or the like. Computing clusters and/or cloud-based computing systems may be used for increased computational power. Such systems may include one or more processors to execute program code (e.g., general-purpose microprocessors usable as a central processing unit (CPU) and/or special-purpose processors such as graphics processors (GPUs) that may provide enhanced parallel-processing capability); memory and other storage devices to store program code and data; user input devices (e.g., keyboards, pointing devices such as a mouse or touchpad, microphones); user output devices (e.g., display devices, speakers, printers); combined input/output devices (e.g., touchscreen displays); signal input/output ports; network communication interfaces (e.g., wired network interfaces such as Ethernet interfaces and/or wireless network communication interfaces such as Wi-Fi); and so on. Computer programs incorporating various features of the present invention may be encoded and stored on various computer readable storage media; suitable media include magnetic disk or tape, optical storage media such as compact disk (CD) or DVD (digital versatile disk), flash memory, and other non-transitory media. (It is understood that “storage” of data is distinct from propagation of data using transitory media such as carrier waves.) Computer readable media encoded with the program code may be packaged with a compatible computer system or other electronic device, or the program code may be provided separately from electronic devices (e.g., via Internet download or as a separately packaged computer-readable storage medium). Input data and/or output data may be provided in secure form, e.g., using blockchain or other encryption technologies.

Thus, although the invention has been described with respect to specific embodiments, it will be appreciated that the invention is intended to cover all modifications and equivalents within the scope of the following claims.

1. A method for modeling virus activity, the method comprising:
for each of a plurality of time periods within an investigation period, determining a quantitative measure of genetic activity of a virus (“g-measure”), wherein the g-measure models a combination of prevalence of effective mutations and number of simultaneous effective mutations; and
using one or more of the g-measure and the prevalence of one or more individual mutations to predict activity of the virus during a future time period subsequent to the investigation period.

2. The method of claim 1 wherein the virus is a flu virus.

3. The method of claim 1 wherein the mutations include mutations in an amino acid sequence of the virus.

4. The method of claim 1 wherein the g-measure is based on data from a particular region and the prediction of activity of the virus is for the particular region.

5. The method of claim 1 wherein the g-measure is based on global data and the prediction of activity of the virus is a global prediction.

6. The method of claim 1 wherein determining the g-measure includes:
obtaining, for each of the time periods within the investigation period, amino acid sequence data for a number of samples of the virus;
determining, based on the amino acid sequence data, a coding sequence for each of the samples of the virus;
determining, for each of the time periods, a prevalence vector based on the coding sequences for each of the samples of the virus, the prevalence vector indicating a prevalence of each amino acid at each sequence position;
identifying, from the prevalence vectors of all of the time periods, one or more effective mutations;
for each effective mutation, identifying an effective mutation period; and
computing the g-measure for each time period based on the effective mutations identified in that time period.

7. The method of claim 6 wherein identifying an effective mutation includes selecting a dominance threshold such that an effective mutation has a prevalence of zero for at least a first time period and a prevalence at least equal to the dominance threshold for at least one time period after the first time period.

8. The method of claim 7 wherein identifying an effective mutation period includes identifying an extended effective mutation period, wherein the effective mutation period includes:
all of the time periods from a first nonzero prevalence of the effective mutation to the earliest time period for which the prevalence of the effective mutation is at least equal to the dominance threshold; and
the extended effective mutation period.

9. The method of claim 8 wherein the dominance threshold and the extended effective mutation period are determined based on optimizing a fit between the g-measure and a population-level epidemic variable indicative of infections caused by the virus during the time periods within the investigation period.

10. The method of claim 6 wherein computing the g-measure for each time period includes computing a sum of the respective prevalences of each effective mutation identified in that time period.

11. The method of claim 6 wherein using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations;
predicting a value of the g-measure for the future time period based on the predicted future prevalence of the one or more individual mutations; and
predicting, based at least in part on the predicted value of the g-measure, a future value of a population-level epidemic variable indicative of infections caused by the virus.

12. The method of claim 6 wherein using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations; and
predicting, based on the predicted future prevalence of the one or more individual mutations, that at least one of the one or more mutations will become dominant in the future time period.

13. The method of claim 12 further comprising:
selecting amino acids to include in a vaccine, wherein the selection includes the at least one of the one or more mutations predicted to become dominant in the future time period.

14. The method of claim 6 wherein using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations; and
defining, for the subsequent time period, a representative viral sequence based on the predicted future prevalence of the one or more individual mutations.

15. The method of claim 14 wherein using one or more of the g-measure and the prevalence of one or more individual mutations to predict activity of the virus during a future time period subsequent to the investigation period further includes:
predicting, based on the prevalence of one or more individual mutations, a future representative strain for a gene segment of a virus.

16. The method of claim 14 further comprising:
selecting, as a viral strain to include in a vaccine, an existing viral strain that is closer to the representative viral sequence for the subsequent time period than any other existing viral strain.

17. The method of claim 6 further comprising:
defining, based on the prevalence vector for a current time period, a representative viral sequence for the current time period;
determining a distance metric between the representative viral sequence and one or more viral strains included in a vaccine; and
determining a likely efficacy of the vaccine based at least in part on the distance metric.

18. A system comprising:
a memory to store data; and
a processor coupled to the memory and configured to:
determine, for each of a plurality of time periods within an investigation period, a quantitative measure of genetic activity of a virus (“g-measure”), wherein the g-measure models a combination of prevalence of effective mutations and number of simultaneous effective mutations; and
use one or more of the g-measure and the prevalence of one or more individual mutations to predict activity of the virus during a future time period subsequent to the investigation period.

19. A computer-readable storage medium having stored thereon program code instructions that, when executed by a processor of a computer system, cause the processor to perform a method comprising:
determining, for each of a plurality of time periods within an investigation period, a quantitative measure of genetic activity of a virus (“g-measure”), wherein the g-measure models a combination of prevalence of effective mutations and number of simultaneous effective mutations; and
using one or more of the g-measure and the prevalence of one or more individual mutations to predict activity of the virus during a future time period subsequent to the investigation period.

20. The system of claim 18 wherein the processor is further configured such that determining the g-measure includes:
obtaining, for each of the time periods within the investigation period, amino acid sequence data for a number of samples of the virus;
determining, based on the amino acid sequence data, a coding sequence for each of the samples of the virus;
determining, for each of the time periods, a prevalence vector based on the coding sequences for each of the samples of the virus, the prevalence vector indicating a prevalence of each amino acid at each sequence position;
identifying, from the prevalence vectors of all of the time periods, one or more effective mutations;
for each effective mutation, identifying an effective mutation period; and
computing the g-measure for each time period based on the effective mutations identified in that time period.

21. The system of claim 20 wherein the processor is further configured such that:
identifying an effective mutation includes selecting a dominance threshold such that an effective mutation has a prevalence of zero for at least a first time period and a prevalence at least equal to the dominance threshold for at least one time period after the first time period;
identifying an effective mutation period includes identifying an extended effective mutation period, wherein the effective mutation period includes:
all of the time periods from a first nonzero prevalence of the effective mutation to the earliest time period for which the prevalence of the effective mutation is at least equal to the dominance threshold; and
the extended effective mutation period; and
the dominance threshold and the extended effective mutation period are determined based on optimizing a fit between the g-measure and a population-level epidemic variable indicative of infections caused by the virus during the time periods within the investigation period.

22. The system of claim 20 wherein the processor is further configured such that using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations; and
defining, for the subsequent time period, a representative viral sequence based on the predicted future prevalence of the one or more individual mutations.

23. The system of claim 22 wherein the processor is further configured such that using one or more of the g-measure and the prevalence of one or more individual mutations to predict activity of the virus during a future time period subsequent to the investigation period further includes:
predicting, based on the prevalence of one or more individual mutations, a future representative strain for a gene segment of a virus.

24. The system of claim 22 wherein the processor is further configured to:
select, as a viral strain to include in a vaccine, an existing viral strain that is closer to the representative viral sequence for the subsequent time period than any other existing viral strain.

25. The computer-readable storage medium of claim 19 wherein determining the g-measure includes:
obtaining, for each of the time periods within the investigation period, amino acid sequence data for a number of samples of the virus;
determining, based on the amino acid sequence data, a coding sequence for each of the samples of the virus;
determining, for each of the time periods, a prevalence vector based on the coding sequences for each of the samples of the virus, the prevalence vector indicating a prevalence of each amino acid at each sequence position;
identifying, from the prevalence vectors of all of the time periods, one or more effective mutations;
for each effective mutation, identifying an effective mutation period; and
computing the g-measure for each time period based on the effective mutations identified in that time period.

26. The computer-readable storage medium of claim 25 wherein using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations;
predicting a value of the g-measure for the future time period based on the predicted future prevalence of the one or more individual mutations; and
predicting, based at least in part on the predicted value of the g-measure, a future value of a population-level epidemic variable indicative of infections caused by the virus.

27. The computer-readable storage medium of claim 25 wherein using one or more of the g-measure and the prevalence of one or more individual mutations to predict activity of the virus during a future time period subsequent to the investigation period includes:
predicting, based on the prevalence of one or more individual mutations and a conditional prevalence distribution that relates prevalence of a mutation in one time period to prevalence in a subsequent time period, a future prevalence of the one or more individual mutations; and
predicting, based on the predicted future prevalence of the one or more individual mutations, that at least one of the one or more mutations will become dominant in the future time period.

28. The computer-readable storage medium of claim 27 wherein the method further comprises:
selecting amino acids to include in a vaccine, wherein the selection includes the at least one of the one or more mutations predicted to become dominant in the future time period.

29. The computer-readable storage medium of claim 25 wherein the method further comprises:
defining, based on the prevalence vector for a current time period, a representative viral sequence for the current time period;
determining a distance metric between the representative viral sequence and one or more viral strains included in a vaccine; and
determining a likely efficacy of the vaccine based at least in part on the distance metric.
